(19) United States
(12) Patent Application Publication
(10) Pub. No.: US 2025/0012769 A1
(43) Pub. Date: Jan. 9, 2025

(54) METHOD AND SYSTEM FOR ASSESSING AND CERTIFYING THE ORIGIN OF HYDROGEN

(71) Applicant: Abu Dhabi National Oil Company, Abu Dhabi (AE)

(72) Inventors: Thierry LUCIDARME, Abu Dhabi (AE); Joao Jorge DA SILVA FERREIRA ALVES, Abu Dhabi (AE)

(73) Assignee: Abu Dhabi National Oil Company, Abu Dhabi (AE)

(21) Appl. No.: 18/707,155

(22) PCT Filed: Dec. 6, 2021
(86) PCT No.: PCT/IB2021/061365

§ 371 Date: May 2, 2024

(51) Int. Cl. G01N 33/00; B01D 53/02; G01N 21/31; G01N 30/02; G01N 30/74

(52) U.S. Cl. CPC G01N 33/005; B01D 53/025; G01N 21/31; G01N 30/74; G01N 33/004; G01N 2030/025

(57) ABSTRACT

Method for assessing and certifying the origin of hydrogen, the method comprising the following steps: a. sampling a quantity of hydrogen gas; b. assessing from the sample results the origin of the hydrogen gas; and c. certifying the origin of the hydrogen gas. In particular, step b. comprises assessing the occurrence of CO, N2, CH4 and/or isotopes, preferably deuterium, in the sample. Further a system for assessing and certifying the origin of hydrogen is claimed.

### 1. FIELD OF THE INVENTION

The invention relates to a method and system for determining and ensuring the origin of hydrogen.

### 2. PRIOR ART

The use of environmentally friendly, low greenhouse gas (GHG) intensity hydrogen is presently considered as one of the key elements in fighting against climate change. Such hydrogen can be used as a substitute for coal, oil or gas in industry applications that cannot directly be powered by electricity.

Thus, in the near future hydrogen is forecast to be one of the main energy sources for the industry.

Hydrogen can be produced by different processes and from different feedstocks and energy sources used. Thus, hydrogen is typically differentiated by its origin, indicating the feedstock, energy source and the production process. The origin of the hydrogen is sometimes indicated by different “colors” of the hydrogen. An exemplary differentiation of the origin of hydrogen and its GHG footprint is shown in Table 1:

TABLE 1

| | | | | Energy | GHG |
|---|---|---|---|---|---|
| | Terminology | Technology | Feedstock | Source | footprint |
| Production | Green Hydrogen | Electrolysis | Water | Wind/Solar/ | Minimal |
| via | | | | Hydro/Geo- | |
| Electricity | | | | thermal/ | |
| | | | | Tidal | |
| | Purple/Pink Hydrogen | | | Nuclear | |
| | Yellow Hydrogen | | | Mixed-origin | Medium |
| | | | | grid energy | |
| Production | Blue Hydrogen | Natural gas reforming + CCUS | Natural Gas/ | various | Low |
| via | | Gasification + CCUS | coal | | |
| Fossil | Turquoise Hydrogen | Pyrolysis | Natural gas | | Solid |
| Fuels | | | | | carbon |
| | | | | | (by- |
| | | | | | product) |
| | Gray Hydrogen | Natural gas reforming | | | Medium |
| | Brown Hydrogen | Gasification | Brown coal | | High |
| | | | (lignite) | | |
| | Black Hydrogen | | Black coal | | |

In the future, hydrogen of various origins will likely be available on the market at various prices. In general, the lower the GHG footprint of the hydrogen, the higher the production cost and the price will be.

Presently, green hydrogen which is produced from water by electrolysis with power from a renewable energy source, like wind, solar, hydro, geothermal or tidal energy, is considered as the most environmentally friendly form of hydrogen. Thus, it has the best GHG footprint as shown in Table 1.

On the other side of the scale black hydrogen that is produced out of black coal by gasification is considered to have a high GHG footprint.

Since the price of the hydrogen of different origins is likely to differ considerably, it is desirable for a customer to ensure that the hydrogen received is of the acquired origin and has the desired GHG footprint. Mixing various types of origins might help to reduce the cost of production, e.g. from 7$/kg to 1$/kg. Therefore, the origin of purchased hydrogen should be monitored.

Further, hydrogen might be delivered from the producer to the consumer through pipelines or classical transportation in high-pressure tanks which do not easily allow differentiation of its origin.

Therefore, it is an object of the present invention to provide a method and a system that enables to verify the origin of hydrogen, in particular to differentiate between hydrogen produced by electrolysis and hydrogen produced directly from fossil fuels.

### 3. SUMMARY OF THE INVENTION

The above-mentioned object is solved by a method for assessing and certifying the origin of hydrogen according to claim 1 and a system for assessing and certifying the origin of hydrogen according to claim 9.

Preferably, the above-mentioned object is solved by a method for assessing and certifying the origin of hydrogen, the method comprising the following steps:
a. sampling a quantity of hydrogen gas;
b. assessing from the sample results the origin of the hydrogen gas; and
c. certifying the origin of the hydrogen gas.

Thus, the method assesses and certifies the origin of hydrogen gas. First, a quantity of hydrogen gas is sampled, preferably measured by different measuring means to obtain certain chemical or physical information about the hydrogen gas. Preferably, the sampling step can be used to determine if a certain chemical element or composition is contained in the gas. Further, from these sampling results the origin of the hydrogen gas is assessed. Preferably, a specific logic can be used to interpret the sampling results to assess the origin of the hydrogen gas with a specific certainty. Finally, the origin of the hydrogen gas is certified. For this certification for example smart certificates for example by using blockchains can be used to ensure correctness of certification.

Preferably, in a first embodiment the step of assessing the origin of the hydrogen gas from the sample comprises:
assessing the occurrence of CO in the sample;
if no CO is contained in the sample, assessing that the origin of the hydrogen is electrolytic;
if CO is contained in the sample, assessing that the origin of the hydrogen is not electrolytic or from a mix of origins.

By such an assessment procedure, a simple and fast assessment of electrolytic hydrogen is possible with some certainty. However, it may be difficult to differentiate electrolysis-produced hydrogen from highly-purified hydrogen from fossil fuels based on CO determination alone. Further, it is possible to detect grey or blue hydrogen but it is difficult to distinguish grey from blue hydrogen. The presence of impurities in hydrogen fuel depends on the feedstock and production process used. Carbon monoxide can be found in hydrogen produced using steam methane reforming or gasification processes. Thus, a CO positive determination in the hydrogen gas sample indicates that the hydrogen is gray or blue. The presence of CO in the hydrogen comes from the steam reforming process. This process can also generate some CO₂ (through the “Water Gas Shift” reaction) which is easier to purify through a “Pressure Swing Adsorption” (PSA) unit. Therefore, the assessment of CO in the sample could be replaced by an assessment of CO₂ in the sample or an assessment of CO and CO₂ in the sample.

Further, since by this assessment procedure the source of electrical energy used for electrolysis production cannot be determined, the electrolytic hydrogen can also be purple/pink hydrogen—made with nuclear power—or yellow hydrogen made with mixed-grid energy, see Table 1.

Preferably, in a second embodiment the step of assessing from the sample the origin of the hydrogen gas comprises:
assessing the occurrence of CO in the sample;
assessing the content of N₂ in the sample;
if no CO is contained in the sample and the content of N₂ in the sample is <1 ppm, assessing that the origin of the hydrogen is electrolytic;
if no CO is contained in the sample and the content of N₂ in the sample is >=1 ppm, assessing that the origin of the hydrogen is not electrolytic or is from a mix of origins; and
if CO is contained in the sample, assessing that the origin of the hydrogen is not electrolytic or is from a mix of origins.

The assessment procedure of the second embodiment improves the certainty in the determination of electrolytic hydrogen. In this embodiment it is possible to differentiate electrolytic hydrogen from more purified hydrogen produced from fossil feedstocks or a mix of electrolytic and fossil-derived hydrogen. Further, it is possible to identify hydrogen from fossil feedstocks but it is not possible to distinguish grey from blue hydrogen. Further, the electrolytic hydrogen can also be purple, pink or yellow hydrogen.

Preferably, in a third embodiment the step of assessing from the sample the origin of the hydrogen gas comprises:
assessing the occurrence of CO in the sample;
assessing the content of N₂ in the sample;
assessing the occurrence of CH₄ in the sample;
if no CO is contained in the sample and the content of N₂ in the sample is <1 ppm and no CH₄ is contained in the sample, assessing that the origin of the hydrogen is electrolytic;
if no CO is contained in the sample and the content of N₂ in the sample is >=1 ppm, assessing that the origin of the hydrogen is not electrolytic or is from a mix of origins;
if no CO is contained in the sample and if CH₄ is contained in the sample, assessing that the origin of the hydrogen is not electrolytic or is from a mix of origins; and
if CO is contained in the sample, assessing that the origin of the hydrogen is not electrolytic or is from a mix of origins.

By the assessment procedure of the third embodiment a further improved assessment of electrolytic hydrogen is possible with better certainty based on the detection of additional impurities, preferably still existing methane (CH₄) in the hydrogen sample. In this embodiment it is possible to differentiate electrolytic hydrogen or a mix of electrolytic hydrogen with hydrogen of other origin with higher certainty. Further, it is possible to detect grey or blue hydrogen but it is not possible to distinguish grey from blue hydrogen. Further, the electrolytic hydrogen can also be purple, pink or yellow hydrogen.

Preferably, in a fourth embodiment the step of assessing from the sample the origin of the hydrogen gas comprises:
assessing the occurrence of CO in the sample;
assessing the content of N₂ in the sample;
assessing the occurrence of CH₄ in the sample;
assessing the content of deuterium in the sample;
if no CO is contained in the sample and the content of N₂ in the sample is <1 ppm and no CH₄ is contained in the sample and the ratio of deuterium to hydrogen in the sample is below a specific threshold, assessing that the origin of the hydrogen is electrolytic;
if no CO is contained in the sample and the content of N₂ in the sample is >=1 ppm and no CH₄ is contained in the sample and the ratio of deuterium to hydrogen in the sample is below a specific threshold, assessing that the origin of the hydrogen is not electrolytic or is from a mix of origins;
if no CO is contained in the sample and the content of N₂ in the sample is <1 ppm and CH₄ is contained in the sample and the ratio of deuterium to hydrogen in the sample is above a specific threshold, assessing that the origin of the hydrogen is not electrolytic or is from a mix of origins;
if no CO is contained in the sample and the content of N₂ in the sample is >=1 ppm and no CH₄ is contained in the sample and the ratio of deuterium to hydrogen in the sample is above a specific threshold, assessing that the origin of the hydrogen is not electrolytic or is from a mix of origins;
if no CO is contained in the sample and the content of N₂ in the sample is >=1 ppm and CH₄ is contained in the sample and the ratio of deuterium to hydrogen in the sample is below a specific threshold, assessing that the origin of the hydrogen is not electrolytic or is from a mix of origins;
if no CO is contained in the sample and the content of N₂ in the sample is >=1 ppm and CH₄ is contained in the sample and the ratio of deuterium to hydrogen in the sample is above a specific deuterium threshold (T_{D}), assessing that the origin of the hydrogen is not electrolytic or is from a mix of origins; and
if CO is contained in the sample, assessing that the origin of the hydrogen is not electrolytic or is from a mix of origins.

By the assessment procedure of the fourth embodiment a further improved assessment of electrolytic hydrogen is possible with better certainty based on the additional detection of isotopes, preferably deuterium. In this embodiment it is possible to differentiate electrolytic hydrogen from a mix of electrolytic hydrogen with hydrogen of other origins with even higher certainty. This embodiment is based on the recognition that isotopes of hydrogen, particularly deuterium, are highly reduced in the hydrogen derived solely from water by electrolysis compared to deuterium in hydrogen derived at least partly from natural gas.

Further, it is possible to detect gray or blue hydrogen but it may not be possible to fully distinguish gray from blue hydrogen. Further, the electrolytic hydrogen can also be purple, pink or yellow hydrogen.

Preferably, the step of sampling of the quantity of hydrogen gas comprises one or more of the following analyses:
chromatography of the sample of hydrogen gas; and/or
spectroscopy of the sample of hydrogen gas; and/or
optical analysis of the hydrogen gas, preferably by laser absorption.

Preferably, the step of sampling of the quantity of hydrogen gas comprises:
CO measurement; and/or
N₂ measurement; and/or
CH₄ measurement; and/or
isotopes measurement, preferably deuterium measurement.

Preferably, the step of certifying the origin of the hydrogen gas is done in an authenticated immutable manner. Thus, the certification step is done preferably fully automatic without the possibility for manipulation.

The above-mentioned problem is also solved by a system for assessing and certifying the origin of hydrogen, wherein the origin of hydrogen describes the of the hydrogen, the system comprises a sampling device, for sampling a quantity of hydrogen gas; an assessing device, for assessing from the sample the origin of the hydrogen; and a certifying device, for certifying the origin of the hydrogen.

Preferably, the sampling device comprises a chromatography device, for performing chromatography of the sample of hydrogen gas; and/or a spectroscopy device, for performing spectroscopy of the sample of hydrogen gas; and/or an optical analysis device, for an optical analysis of the hydrogen gas.

Preferably, the optical analysis device comprises a laser absorption device.

Preferably, the chromatography device comprises a gas chromatography device coupled with pulsed discharge helium ionization detector (GC-PDHID) or a gas chromatography coupled with thermal conductivity detector (GC-TCD). The gas chromatograph coupled with a pulsed discharge helium ionization detector (GC-PDHID) relies on the ionization of compounds by a plasma of helium (done by a pulsed DC discharge). In a GC-TCD gas chromatography device a thermal conductivity detector (TCD) measures the difference of thermal conductivity between the pure carrier gas (usually hydrogen or helium) and the carrier gas with sample compounds with a Wheatstone bridge. The temperature of a filament changes in presence of the sample compounds, leading to a variation of the resistance and of the voltage.

A GC-PDHID gas chromatography device or a GC-TCT gas chromatography device is particularly capable to measure the amount of nitrogen, methane and carbon monoxide in hydrogen.

An optical analysis device preferably comprises an optical spectroscopy device. Optical spectroscopic methods are an alternative to chromatographic methods for hydrogen analysis. In these methods, the intensity of light at a specific wavelength after absorption by matter is measured. The wavelength is typically selected to be specific to the selected chemical compound but some interference might exist. Several different spectroscopic methods can be used including cavity ring-down spectroscopy (CRDS), optical feedback cavity enhanced absorption spectroscopy (OFCEAS), Fourier transform infrared spectroscopy (FTIR), and laser-based direct absorption spectroscopy (DAS).

In CRDS, the light source is a laser emitting at a specific wavelength in an optical resonator composed of two reflective mirrors. The decay time until the light reaches a fraction of its initial intensity is measured. This time decreases when the concentration of the compound absorbing the wavelength increases. This technique allows sensitive detection, down to ppt, due to a high path length of typically several kilometers. However, the analytes which can be measured are limited by the available laser and mirrors wavelengths.

Optical Feedback Cavity Enhanced Absorption Spectroscopy (OFCEAS) is a variant of CRDS with a long optical path at low pressure, below 100 mbar absolute, enabling sampling at low pressure and a reduction of the needed sample volume. Analysis over a range of wavelengths can be performed quickly. With OFCEAS it is particularly possible to optically measure methane and carbon monoxide in hydrogen.

The paper Review and Survey of Methods for Analysis of Impurities in Hydrogenfor Fuel Cell Vehicles According to ISO 14687:2019 of Beurey C, Gozlan B, Carré M, Bacquart T, Morris A, Moore N, Arrhenius K, Meuzelaar H, Persijn S, Rojo A and Murugan A., Frontiers in Energy Research, February 2021, Volume 8, Article 615149 describes various methods of measuring impurities in hydrogen for fuel cell vehicle applications.

A hydrogen isotope analysis to determine the deuterium (D) content in the hydrogen can be preferably done with a gas chromatography combustion isotope ratio mass spectrometry (GC/C/IRMS) device.

Preferably, the sampling device is capable to perform CO measurement and/or N₂ measurement and/or CH₄ measurement; and/or isotopes measurement, preferably deuterium measurement.

It can be noted here that the detection process according to the invention can be circumvented, if the of the hydrogen is not electrolytic, by means of increased purification of the hydrogen produced in a process using fossil feedstocks to lower the level of impurities. However, this increases the cost of the produced hydrogen and creates a disincentive for such adulteration. Therefore, this further justifies the present invention.

Preferably, depending on the level of certainty of assessment the following measurements are made:
CO measurement, or
CO and N2 measurement, or
CO, N2 and CH4 measurement, or
CO, N2, CH4 and isotopes measurement, preferably deuterium measurement.

Therefore, an adaptive system is provided that depending on the desired level of certainty or doubt adds further measuring steps to the sampling of the sample of hydrogen gas.

Preferably, the certifying device provides a digital certificate in an authenticated immutable manner, preferably in form of a blockchain.

The above mentioned problem is also solved by a system as described above for performing the method as described above.

### 4. SHORT DESCRIPTION OF THE DRAWINGS

In the following, preferred embodiments of the invention are disclosed by reference to the accompanying figure, in which shows:

FIG. 1 a schematic overview of a method for assessing and certifying the origin of hydrogen; and

FIG. 2 a schematic overview of a system for assessing and certifying the origin of hydrogen.

### 5. DETAILED DESCRIPTION OF PREFERRED EMBODIMENTS

In the following, preferred embodiments of the invention are described in detail with respect to the figures.

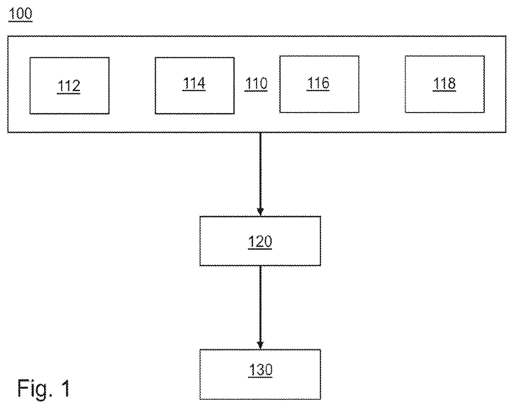

FIG. 1 shows schematic overview of a method for assessing and certifying the origin of hydrogen 100. The method 100 comprising the following steps:

a. sampling a quantity of hydrogen gas 110;
b. assessing from the sample results the origin of the hydrogen gas 120; and
c. certifying the origin of the hydrogen gas 130.

The sampling step 110 may comprise the sub-steps of:
assessing the occurrence of CO in the sample 112;
assessing the concentration of N2 in the sample 114;
assessing the occurrence of CH₄ in the sample 116;
assessing the distribution of deuterium in the sample 118.

In a first embodiment the sampling step 110 comprises the sub-step of assessing the occurrence of CO in the sample 112. Based on such a sampling the origin of the hydrogen gas can be assessed according the logic of Table 2:

TABLE 2

| CO | Origin |
|---|---|
| 0 | Electrolytic |
| 1 | Not electrolytic or |
| | mix of origins |

In a second embodiment, the sampling step 11o comprises the sub-steps of assessing the occurrence of CO in the sample 112 and assessing the content of N₂ in the sample 114. Based on such a sampling the origin of the hydrogen gas can be assessed according the logic of Table 3:

TABLE 3

| CO | N₂ < 1 ppm | N₂ >= 1 ppm | Origin |
|---|---|---|---|
| 0 | 1 | 0 | Electrolytic |
| 0 | 0 | 1 | Not electrolytic or |
| | | | mix of origins |
| 1 | any | any | Not electrolytic or |
| | | | mix of origins |

In a third embodiment, the sampling step 110 comprises the sub-steps of assessing the occurrence of CO in the sample 112, assessing the content of N_{Z} in the sample 114, and assessing the occurrence of CH₄ in the sample. Based on such a sampling the origin of the hydrogen gas can be assessed according the logic of Table 4:

TABLE 4

| | N₂ < | N₂ >= | | |
|---|---|---|---|---|
| CO | 1 ppm | 1 ppm | CH₄ | Origin |
| 0 | 1 | 0 | 0 | Electrolytic |
| 0 | 0 | 1 | any | Not electrolytic or mix of |
| | | | | origins |
| 0 | 0 | any | 1 | Not electrolytic or mix of |
| | | | | origins |
| 1 | any | any | any | Not electrolytic or mix of |
| | | | | origins |

In a fourth embodiment, the sampling step 110 comprises the sub-steps of assessing the occurrence of CO in the sample 112, assessing the content of N₂ in the sample 114, assessing the occurrence of CH₄ in the sample, and assessing the ratio of deuterium to hydrogen in the sample. Based on such a sampling the origin of the hydrogen gas can be assessed according the logic of Table 5:

TABLE 5

| | N₂ < | N₂ >= | | D < | D >= | |
|---|---|---|---|---|---|---|
| CO | 1 ppm | 1 ppm | CH₄ | T_{D} | T_{D} | Origin |
| 0 | 1 | 0 | 0 | 1 | 0 | Electrolytic |
| 0 | 0 | 1 | 0 | 1 | 0 | Not electrolytic or |
| | | | | | | mix of origins |
| 0 | 1 | 0 | 1 | 0 | 1 | Not electrolytic or |
| | | | | | | mix of origins |
| 0 | 0 | 1 | 0 | 0 | 1 | Not electrolytic or |
| | | | | | | mix of origins + |
| 0 | 0 | 1 | 1 | 1 | 0 | Not electrolytic or |
| | | | | | | mix of origins+ |
| 0 | 0 | 1 | 1 | 0 | 1 | Not electrolytic or |
| | | | | | | mix of origins ++ |
| 1 | any | any | any | any | any | Not electrolytic or |
| | | | | | | mix of origins |

Where + and ++ indicate increased certainty in the determination that the hydrogen does not originate from electrolysis or is from multiple origins.

The threshold T_{D} of deuterium D in the hydrogen be preferably a value in the range of 30-140 ppmₘₒₗ, 40-100 ppmₘₒₗ, 50-75 ppmₘₒₗ or similar as determined for the specific location and application, with T_{D} being defined as the ratio of number of mol of deuterium to the number of mol of hydrogen (protium). Other ratio can also be used for instance normalized ratio of deuterium/hydrogen proportion compared to the deuterium/hydrogen proportion in see water.

Preferred specific deuterium thresholds T_{D} may be 30, 40, 50, 60, 70, 80, 90, 100, 110 or 120 ppmₘₒₗ. Particularly preferred is a deuterium threshold of 50 ppmₘₒₗ. As per the article “Deuterium separation by combined Electrolysis Fuel cell, Energy (2018), doi:10.1016/j.energy.2018.02.014”, the deuterium content of hydrogen originating from electrolysis is significantly reduced (divided roughly per 8) compared to the deuterium content of water itself (150 ppmₘₒₗ/8=19 ppmₘₒₗ) whereas the deuterium content of hydrogen originating from fossil feedstocks contains a higher proportion of deuterium coming from the fossil feedstock itself and from water (approximately 150 ppmₘₒₗ) through the water gas shift reaction.

Preferably, depending on the degree of certainty that is desired with respect to the origin of the hydrogen, further rare impurities generated by fossils processes like Helium, Arsenic, Argon can be determined to enforce the probability of detecting not electrolytic hydrogen.

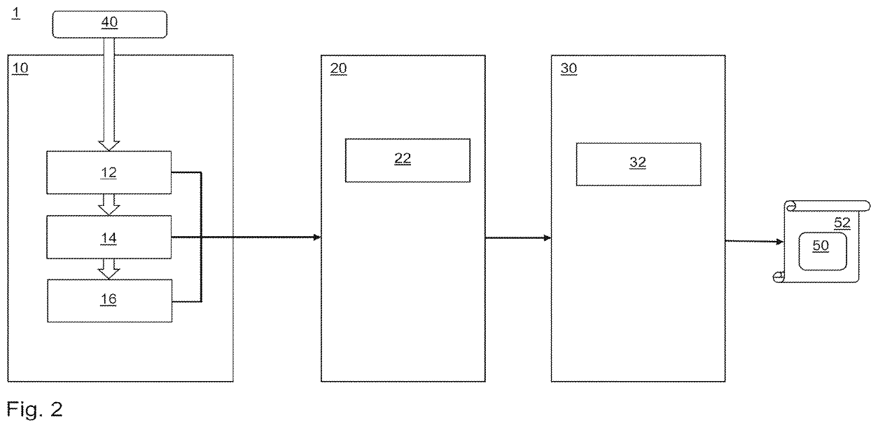

The certifying step 130 certifies the origin of the assessed hydrogen preferably in an authenticated immutable manner. Thus, as digital smart certificate 50, for example in the form of a blockchain 52 may be used for such a certification (see FIG. 2). In the blockchain preferably all information relating to the batch of hydrogen, for example a batch number, quantity, date of production, manufacturer, manufacturing site, certifier and the origin of the hydrogen are stored. Further, analysis results, like the content of CO, N₂, CH₄, deuterium or other hydrogen impurities or characteristics may be stored within the smart certificate 50.

FIG. 2 shows a system for assessing and certifying the origin of hydrogen 1. The system 1 comprises a sampling device 10, for sampling a quantity of hydrogen gas 40, an assessing device 20, for assessing from the sample the origin of the hydrogen gas and a certifying device 30, for certifying the origin of the hydrogen gas.

The sampling device 10 receives a quantity of hydrogen gas 40 and performs measurements on the hydrogen gas 40 to determine impurities that are characteristic for the or origin of the hydrogen.

To perform such measurements the sampling device 10 may comprise a chromatography device 12, for performing chromatography of the sample of hydrogen gas. Preferably the chromatography device 12 is a GC-PDHID gas chromatography device or a GC-TCT gas chromatography device, as such gas chromatography devices are capable to measure the amount of nitrogen, methane and carbon monoxide in the hydrogen sample 40.

Further, the sampling device 10 may comprise a spectroscopy device 14, for performing spectroscopy of the sample of hydrogen gas. Several different spectroscopic methods can be used including cavity ring-down spectroscopy (CRDS), optical feedback cavity enhanced absorption spectroscopy (OFCEAS), Fourier transform infrared spectroscopy (FTIR).

Further, the sampling device may comprise an optical analysis device 16, for an optical analysis of the hydrogen gas. Such an optical analysis device 16 may comprise a laser absorption device. Particularly, a laser-based direct absorption spectroscopy (DAS) device can be used to sample the hydrogen for impurities.

The sampling results are transmitted from the sampling device 10 to the assessing device 20. The assessing device comprises at least one calculation means 22, which can be a processor, computer, cloud computing device, or the like, which interprets the sampling results and determines the origin of the hydrogen according a specific logic as shown in the Tables 2 to 4.

The origin of the sampled hydrogen 40 is transmitted from the assessing device 20 to the certifying device 30. The certifying device 30 also comprises at least one calculation means 32, which can be a processor, computer, cloud computing device, or the like. The certifying device 30 certifies the origin of the hydrogen gas, by providing a digital certificate in an authenticated immutable manner. Preferably, certifying device 30 calculates the digital smart certificate 50 which is contained within an electronic data file, for example in the form of a blockchain 52.

1. A method for assessing and certifying the origin of hydrogen, the method comprising the following steps:
a. sampling a quantity of hydrogen gas;
b. assessing from the sample results the origin of the hydrogen gas; and
c. certifying the origin of the hydrogen gas.

2. The method according to claim 1, wherein the step of assessing from the sample the origin of the hydrogen gas comprises:
assessing the occurrence of CO in the sample;
if no CO is contained in the sample, assessing that the origin of the hydrogen is electrolytic; and
if CO is contained in the sample, assessing that the origin of the hydrogen is not electrolytic or from a mix of origins.

3. The method according to claim 1, wherein the step of assessing from the sample the origin of the hydrogen gas comprises:
assessing the occurrence of CO in the sample;
assessing the content of N₂ in the sample;
if no CO is contained in the sample and the content of N₂ in the sample is <1 ppm, assessing that the origin of the hydrogen is electrolytic;
if no CO is contained in the sample and the content of N₂ in the sample is >=1 ppm, assessing that the origin of the hydrogen is not electrolytic or is from a mix of origins; and
if CO is contained in the sample, assessing that the origin of the hydrogen is not electrolytic or is from a mix of origins.

4. The method according to claim 1, wherein the step of assessing from the sample the origin of the hydrogen gas comprises:
assessing the occurrence of CO in the sample;
assessing the content of N₂ in the sample;
assessing the occurrence of CH₄ in the sample;
if no CO is contained in the sample and the content of N₂ in the sample is <1 ppm and no CH₄ is contained in the sample, assessing that the origin of the hydrogen is electrolytic;
if no CO is contained in the sample and the content of N₂ in the sample is >=1 ppm, assessing that the origin of the hydrogen is not electrolytic or is from a mix of origins;
if no CO is contained in the sample and if CH₄ is contained in the sample, assessing that the origin of the hydrogen is not electrolytic or is from a mix of origins; and
if CO is contained in the sample, assessing that the origin of the hydrogen is not electrolytic or is from a mix of origins.

5. The method according to claim 1, wherein the step of assessing from the sample the origin of the hydrogen gas comprises:
assessing the occurrence of CO in the sample;
assessing the content of N₂ in the sample;
assessing the occurrence of CH₄ in the sample;
assessing the content of deuterium in the sample;
if no CO is contained in the sample and the content of N₂ in the sample is <1 ppm and no CH₄ is contained in the sample and the content of deuterium in the sample is below a specific deuterium threshold (T_{D}), assessing that the origin of the hydrogen is electrolytic;
if no CO is contained in the sample and the content of N₂ in the sample is >=1 ppm and no CH₄ is contained in the sample and the content of deuterium in the sample is below a specific deuterium threshold (T_{D}), assessing that the origin of the hydrogen is not electrolytic or is from a mix of origins;
if no CO is contained in the sample and the content of N₂ in the sample is <1 ppm and CH₄ is contained in the sample and the content of deuterium in the sample is above a specific deuterium threshold (T_{D}), assessing that the origin of the hydrogen is not electrolytic or is from a mix of origins;
if no CO is contained in the sample and the content of N₂ in the sample is >=1 ppm and no CH₄ is contained in the sample and the content of deuterium in the sample is above a specific deuterium threshold (T_{D}), assessing that the origin of the hydrogen is not electrolytic or is from a mix of origins;
if no CO is contained in the sample and the content of N₂ in the sample is >=1 ppm and CH₄ is contained in the sample and the content of deuterium in the sample is below a specific deuterium threshold (T_{D}), assessing that the origin of the hydrogen is not electrolytic or is from a mix of origins;
if no CO is contained in the sample and the content of N₂ in the sample is >=1 ppm and CH₄ is contained in the sample and the content of deuterium in the sample is above a specific deuterium threshold (T_{D}), assessing that the origin of the hydrogen is not electrolytic or is from a mix of origins; and
if CO is contained in the sample, assessing that the origin of the hydrogen is not electrolytic or is from a mix of origins.

6. The method according to claim 1, wherein the step of sampling of the quantity of hydrogen gas comprises one or more of the following analyses:
chromatography of the sample of hydrogen gas; and/or
spectroscopy of the sample of hydrogen gas; and/or
optical analysis of the hydrogen gas, preferably by laser absorption.

7. The method according to claim 1, wherein the step of sampling of the quantity of hydrogen gas comprises:
CO measurement; and/or
N₂ measurement; and/or
CH₄ measurement; and/or
isotopes measurement, preferably deuterium measurement.

8. The method according to claim 1, wherein the step of certifying the origin of the hydrogen gas is done in an authenticated immutable manner.

9. A system for assessing and certifying the origin of hydrogen, the system comprises:
a. a sampling device, for sampling a quantity of hydrogen gas;
b. an assessing device, for assessing from the sample the origin of the hydrogen gas; and
c. a certifying device, for certifying the origin of the hydrogen gas.

10. The system according to claim 9, wherein the sampling device comprises
a chromatography device, for performing chromatography of the sample of hydrogen gas; and/or
a spectroscopy device, for performing spectroscopy of the sample of hydrogen gas; and/or
an optical analysis device, for an optical analysis of the hydrogen gas.

11. The system according to claim 10, wherein the optical analysis device comprises a laser absorption device.

12. The system according to claim 10, wherein the chromatography device comprises a gas chromatography device coupled with pulsed discharge helium ionization detector (GC-PDHID) or a gas chromatography device coupled with thermal conductivity detector (GC-TCD).

13. The system according to claim 9, wherein the sampling device is capable to perform CO measurement and/or N₂ measurement and/or CH₄ measurement and/or isotopes measurement, preferably deuterium measurement.

14. The system according to claim 13, wherein depending on the level of certainty of assessment the following measurements are made:
CO measurement, or
CO and N₂ measurement, or
CO, N₂ and CH₄ measurement, or
CO, N₂, CH₄ and isotopes measurement, preferably deuterium measurement.

15. The system according to claim 9, wherein the certifying device provides a digital certificate in an authenticated immutable manner, preferably in form of a blockchain.

16. (canceled)
